(19) United States
(12) Patent Application Publication
(10) Pub. No.: US 2003/0190303 A1
(43) Pub. Date: Oct. 9, 2003

(54) LACTOFERRIN AS REGULATOR OF ALLERGEN-INDUCED TUMOR NECROSIS FACTOR-ALPHA PRODUCTION AND THERAPEUTIC APPLICATIONS

(76) Inventors: IAN KIMBER, CHESHIRE (GB); MARIE CUMBERBATCH, CHESHIRE (GB); REBECCA J. DEARMAN, CHESHIRE (GB); ORLA M. CONNEELY, HOUSTON, TX (US); PAULINE WARD, HOUSTON, TX (US)

Correspondence Address: FULBRIGHT & JAWORSKI, LLP, 1301 MCKINNEY, SUITE 5100, HOUSTON, TX, 77010-3095, US

(21) Appl. No.: 09/058,589

(22) Filed: Apr. 10, 1998

Related U.S. Application Data
(60) Provisional application No. 60/041,890, filed on Apr. 10, 1997.

(51) Int. Cl. A61K 38/16; A61K 38/00; A61K 31/74; A61K 45/00; A61K 31/47

(52) U.S. Cl. 424/78.05; 514/8; 514/12; 514/825; 514/826; 514/844; 514/863; 514/864; 424/85.2; 424/78.03; 530/395; 530/400

(57) ABSTRACT

The present invention relates to pharmaceutical compositions and methods for treating allergic disorders characterized by a local immune response including inflammatory skin reactions, asthma, and arthritis.

### FIELD OF THE INVENTION

The present invention relates to pharmaceutical compositions and methods for the treatment of disorders related to inappropriate or exaggerated immune reactions in response to an allergen, including inflammatory skin reactions, asthma, and arthritis.

### BACKGROUND OF THE INVENTION

Lactoferrin (LF) is an 80 kilo Dalton (kD) iron-binding glycoprotein found in high concentrations in milk and in lower concentrations in other secretions and body fluids. It is one of a number of iron binding proteins, referred to as transferring, involved in iron binding and delivery in mammals. Montreuil and Mullet, 1960, C.R. Acad. Sci. Paris 250:1736-1737; Montreuil et al., 1960, Biochem. Biophys. Acta 45:413-421; Johansson, 1960, Acta Chem. Scand. 14:510-512, Blanc and Isliker, 1961, Bull. Soc. Chim. Biol. 43:929-943; Masson and Heremans, 1967, Protides Biol. Fluids Proc. Colloq. 21:115-124; Querinjenn et al., 1971, Eur. J. Biochem. 20:420-425; Leger et al., 1977, Biol. Anim. Biochim. Biophys. 17:737-747.

Lactoferrin was originally discovered in milk where it can reach levels of 7 grams/liter in colostrum. Since then, however, it has been detected in a number of other body fluids including tears, saliva and mucosal secretions and also in the secondary granules of polymorphonuclear leukocytes. Biserte et al., 1963, Exp. Ann. Biochim. Med. 25:85-120; Masson, 1970, in: La Lactoferrine, pp. 93-165, Arscia, Bruxelles. Thus, the protein is expressed primarily by glandular epithelial cells and neutrophils associated with both local and central immune defense.

Lactoferrin has been shown to play important roles in host defense mechanisms due to its well-established antimicrobial activities. The antimicrobial actions of lactoferrin appear at least in part to be the consequence of the iron binding properties of lactoferrin via sequestration of iron necessary for microbial growth.

It has been shown that lactoferrin production is induced by lipopolysaccharides (LPS), which are components of bacterial cell walls. Gutteberg et al., 1990, Scan. J. Clin. Lab. Invest. 50:421-427. There is mounting evidence that in addition to antimicrobial activities, lactoferrin may influence innate and adaptive immune processes, including natural killer cell function, participate in the course of inflammation, and complement activation and affect cytokine production. Lash et al., 1983, Blood 61:885-888; Mansson et al., 1990, Ann. Rheum. Dis. 49:594-597; Van Snick et al., 1974, J. Exp. Med. 140:1068-1084. With respect to the latter it has been demonstrated, both in vivo and in vitro, that lactoferrin may compromise the production of tumor necrosis factor a (TNF-α) systemically (Machnicki et al., 1993, Int. J. Exp. Path. 74:433-439), a cytokine that plays important roles in inflammation, sepsis and endotoxemic shock (Beutler et al., 1985, Science 229:869-871; Tracey et al., 1987, Curr. Opinion Immunol. 1:454-461; Waage et al., 1989, J. Exp. Med. 169:333-338; Kunkel et al., 1989, Crit. Rev. Immunol. 9:93-117).

In previous investigations, however, attention focused upon the regulation of TNF-α production provoked by LPS. Machnicki et al., 1993, Int. J. Exp. Path. 74:433-439; Gutteberg et al., 1990, APMIS 98: 1027-1032; and Gutteberg et al., 1991, APMIS 99:602-608. Since lactoferrin is known to bind directly to LPS, it was uncertain whether the observed reduction in TNF-α was attributable to transcriptional or post-transcriptional regulation of the cytokine itself or to the reduction by lactoferrin in the availability of LPS due to direct binding and inactivation of the endotoxin. Ellison and Giehl, 1991, J. Clin. Invest. 88:1080-1091; Appelmelk et al., 1994, Infect. Immunity 62:2628-2632. There has not been a previous demonstration of the allergen-induced effects of lactoferrin. For this reason, experiments have now been performed to examine the ability of homologous recombinant lactoferrin to influence the induction of TNF-α-dependent biological responses where stimulation of TNF-α expression is independent of LPS.

A cDNA encoding lactoferrin and methods for recombinantly producing the same have been disclosed by Conneely et al. in U.S. Pat. Nos. 5,571,896, 5,571,697 and 5,571,691. Production of lactoferrin as a fusion product has been disclosed in U. S. Ser. Nos. 08/453,703, 08/456,106 both filed May 30, 1995 and Ser. No. 08/691,123 filed on Aug. 1, 1996. Moreover, the use of lactoferrin to modulate or neutralize heparin activity has been disclosed in U.S. Ser. No. 08/391,986 filed on Feb. 21, 1995. Additionally, lactoferrin mutants and variants thereof have been disclosed in U.S. Ser. No. 08/866,544 filed on May 30, 1997. The disclosures of all the foregoing patents and applications are herein incorporated by reference in their entirety.

### SUMMARY OF THE INVENTION

Lactoferrin suppresses the induction of allergen dependent inflammatory responses. The present invention demonstrates that lactoferrin may inhibit TNF-α dependent responses that are not induced by an endotoxin, i.e., the lipopolysaccharide (LPS) component of the bacterial cell wall. Lactoferrin is an active compound for the treatment of a variety of inflammatory disorders that are the consequence of local immune reactions initiated by allergenic agents.

One aspect of the present invention features compositions suppressing local inflammatory reactions. The compositions comprises a lactoferrin and a pharmaceutically acceptable carrier. These compositions may alternatively, or in addition, include functional analogs or functional fragments of lactoferrin which exhibit the desired inhibitory activities on the locally induced TNF-α-dependent inflammation.

In another aspect, the invention provides methods for treating diseases characterized by a local immune reaction in a mammal by administering an effective amount of a composition comprising a lactoferrin and a pharmaceutically acceptable carrier. Compositions useful for the treatment methods of the present invention may alternatively, or in addition, include functional analogs or functional fragments of lactoferrin that exhibit inhibitory activity on the locally induced TNF-α dependent inflammatory reactions.

The compositions and methods of the present invention are useful for treating a variety of indications that are the consequence of local immune reactions. Such indications include inflammatory skin disorders such as contact allergic dermatitis, psoriasis, UV-induced inflammation, infant diaper rash, pulmonary inflammatory diseases and acne.

Further, the compositions and methods of the present invention may be used as a supplement in anti-wrinkle cosmetic solutions to eliminate the inflammatory side effects of hydroxyacids, which are the most frequently used active ingredient in such cosmetic products.

Finally, in another aspect, the compositions and methods of the present invention may be employed for the treatment of other indications involving local immune responses resulting in inflammation. Such indications include, but are not limited to, asthma and arthritis.

### BRIEF DESCRIPTION OF THE DRAWINGS

FIG. 1A depicts a cell migration cascade in the skin which results in a local immune response and inflammation.

FIG. 1B depicts an interaction of cytokines produced by Langerhans cells and keratinocytes in the epidermis which triggers a local immune response in the skin.

FIG. 2 depicts the binding of lactoferrin to its receptors on TNF-α producing keratinocytes in situ in neonatal mouse skin. The top two panels show the bright and the dark field images obtained using only labelled lactoferrin. The bottom two panels show the bright and the dark field images when the binding assay was performed in the presence of excess unlabelled lactoferrin.

FIG. 3A demonstrates that intradermal administration of lactoferrin inhibits the accumulation of dendritic cells in draining lymph nodes induced by oxazolone, but not that stimulated by injection of TNF-α.

FIG. 3B demonstrates that intradermal administration of lactoferrin inhibits both oxazolone and IL-1β-induced accumulation of dendritic cells in draining lymph nodes.

FIG. 4 demonstrates the influence of lactoferrin on TNF-α and IL-1β-induced Langerhans cell migration.

FIG. 5 provides results from two independent experiments (panels A and B) indicate that treatment with oxazolone induced accumulation of dendritic cells in draining lymph nodes of mice pretreated with 0.02% BSA (see bar 2 panels A and B versus bar 1 control). Topical administration of mouse lactoferrin results in a strong inhibition of accumulation of dendritic cells in the lymph nodes in response to oxazolone in both experiments (see bar 3, panels A and B). The results indicate that topically administered lactoferrin is highly effective at inhibiting dendritic cell accumulation in lymph nodes in response to an allergen and hence allergen-induced cutaneous inflammation.

### DETAILED DESCRIPTION OF THE INVENTION

### Regulation of Tumor Necrosis Factor a (TNF-α) Production by Lactoferrin

The present invention is based on the surprising discovery that lactoferrin and analogs and derivatives thereof including truncated molecules, biologically active fragments and muteins having substitutions and/or deletions are capable of inhibiting a variety of responses that require the production of TNF-α by endotoxin (LPS) independent pathways.

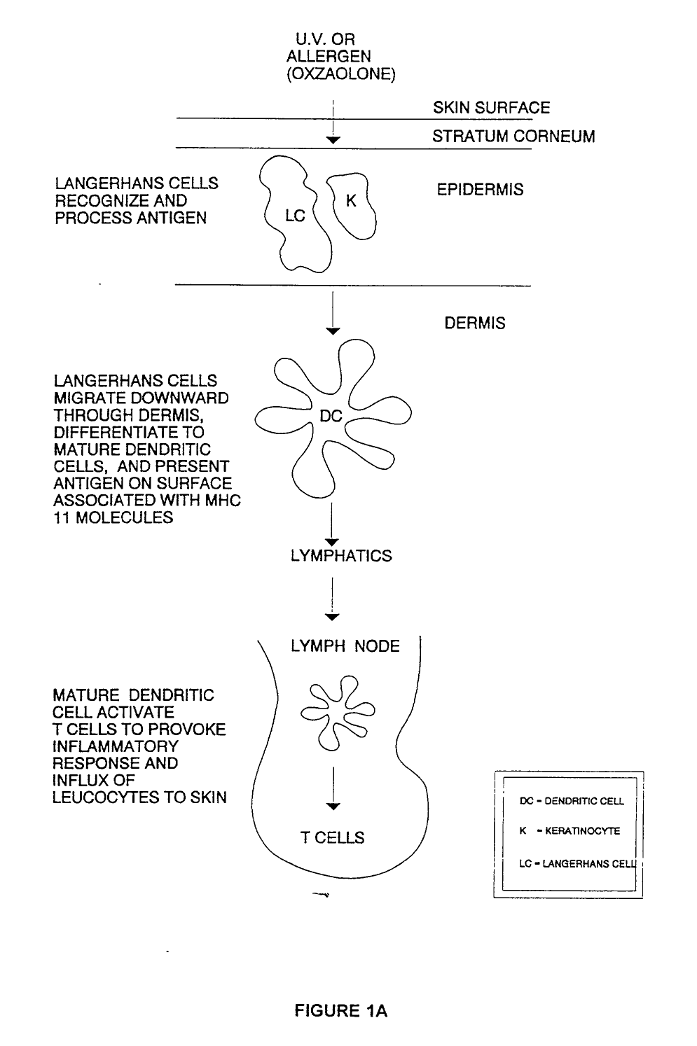

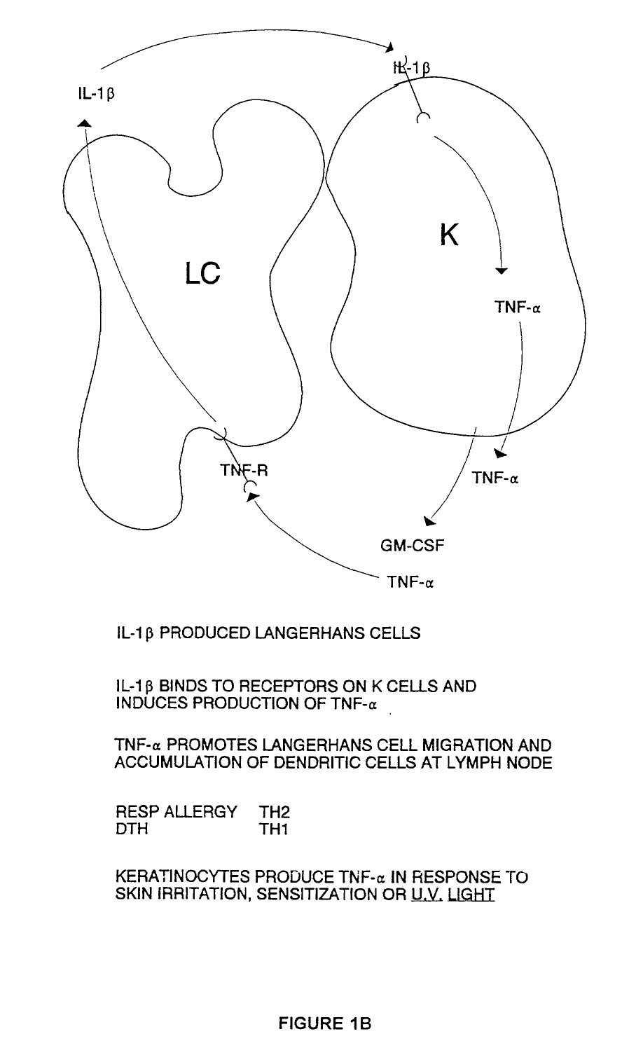

Inflammatory disorders are frequently the consequence of local immune reactions, e.g., caused by exposure to chemicals including exogenously and endogenously produced chemicals or allergens, or triggered by an autoimmune response. Such an inflammatory reaction is caused by trapping of the antigen by immune “Langerhans” cells that are localized in the epidermis. Once the antigen is trapped, the Langerhans cells undergo a maturation or differentiation process that allows them to dissociate from the epidermis and migrate as mature dendritic cells (DCs) downward through the dermis of the skin to the lymphatic system. They may then be carried via the lymphatic system to the draining lymph nodes where they present processed antigen on their cell surface to the T-lymphocytes. Kimber and Cumberbatch, 1992, Toxicol. Appl. Pharmacol. 117:137-146. See, FIG. 1A. The activated T-cells may respond by triggering a complex immune reaction that results in invasion of the skin by inflammatory white blood cells to elicit a local inflammatory response.

Thus, local immune reactions, e.g., cutaneous immunity, requires:

(1) recognition and trapping of an antigen by Langerhans cell maturation and migration of dendritic cells to a lymph node;

(2) activation of T-cells in the lymph node by an antigen presenting dendritic cell; and

(3) subsequent stimulation of inflammatory white blood cell recruitment to the skin.

Each of these processes is dependent upon one or more cytokines that serve to regulate the function of the participating immune cells.

Cytokines that initiate an inflammatory response such as the cutaneous inflammatory response cascade are produced by both keratinocyte and Langerhans cells of the epidermis. Langerhans cells are localized in the epidermis in close apposition to keratinocyte cells as is evident from FIG. 1B.

The induction of Langerhans cell migration by chemical allergens and the subsequent accumulation of dendritic cells in draining lymph nodes is dependent upon the local availability of TNF-α, a cytokine produced in the epidermis by keratinocytes in response to skin sensitization or other forms of dermal trauma. Cumberbatch and Kimber, 1992, Immunology 75:257-263; Cumberbatch et al., 1994, Immunology 81:395-401; Cumberbatch and Kimber, 1995, Immunology 84:31-35. Systemic administration of a neutralizing anti-TNF-α antibody almost completely inhibits the stimulation of Langerhans cell migration and dendritic cell accumulation induced by chemicals such as oxazolone. Cumberbatch and Kimber, 1995, supra. Intradermal administration of homologous recombinant TNF-α is itself sufficient to induce migration of Langerhans cells from the skin and to cause an increase in the number of dendritic cells found within draining lymph nodes. Cumberbatch and Kimber, 1992, supra; Cumberbatch et al., 1994, supra.

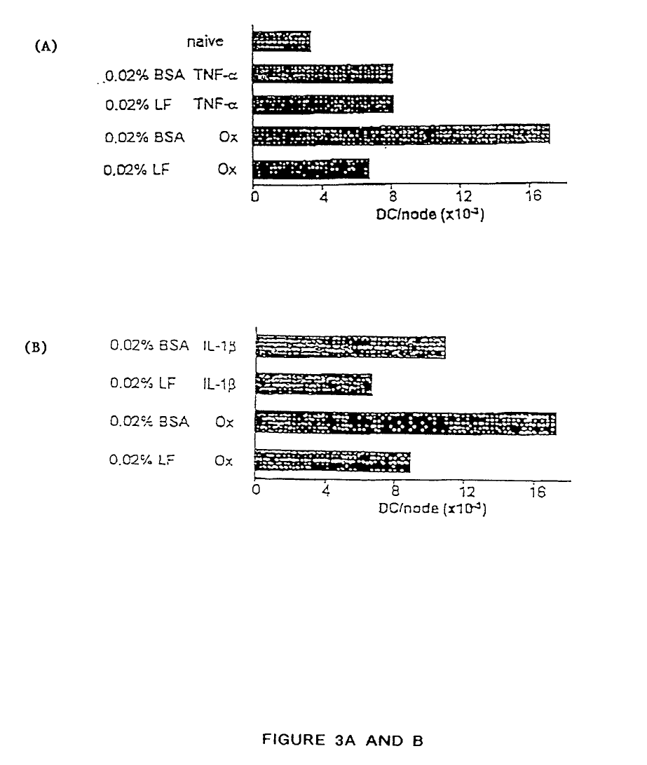

Intradermal administration of lactoferrin and functional analogs and functional fragments thereof inhibits the accumulation of dendritic cells in draining lymph nodes induced by oxazolone, but not that stimulated by injection of TNF-α as demonstrated by FIG. 3A and Examples 2 and 3. These data are consistent with the negative regulation by lactoferrin of the production of TNF-α by keratinocytes that is known to be stimulated following skin sensitization.

It has previously been demonstrated that the stimulation of Langerhans cell migration and dendritic cell accumulation is dependent also upon the local availability of another epidermal cytokine, interleukin 1β (IL-1β). Enk et al., 1993, J. Immunol. 150:3698-3704. This cytokine is produced constitutively in small amounts in murine epidermis by Langerhans cells and its expression is upregulated further as the result of skin sensitization. Enk and Katz, 1992, Proc. Natl. Acad. Sci. 89:1398; Enk and Katz, 1992, J. Invest. Dermatol. 99:S39-S41. Systemic administration of a neutralizing anti-IL-1β antibody inhibits Langerhans cell migration and dendritic cell accumulation. Enk et al., 1993, supra.

It is clear, therefore, that effective Langerhans cell migration is dependent upon the availability of IL-1β and the de novo synthesis by keratinocytes of TNF-α. This is further evidenced by previous work demonstrating that intradermal administration of IL-1β alone induces Langerhans cell migration. See, FIG. 3B and Examples 2 and 3.

Lactoferrin inhibits both oxazolone-induced and IL-1β-induced dendritic cell accumulation in draining lymph nodes. Thus, lactoferrin impairs IL-1β-induced dendritic cell accumulation, but not that induced by TNF-α (see, FIG. 3A). This demonstrates that the inhibitory effect of lactoferrin is downstream of IL-1β and upstream of TNF-α in the cytokine cascade triggering the local immune response.

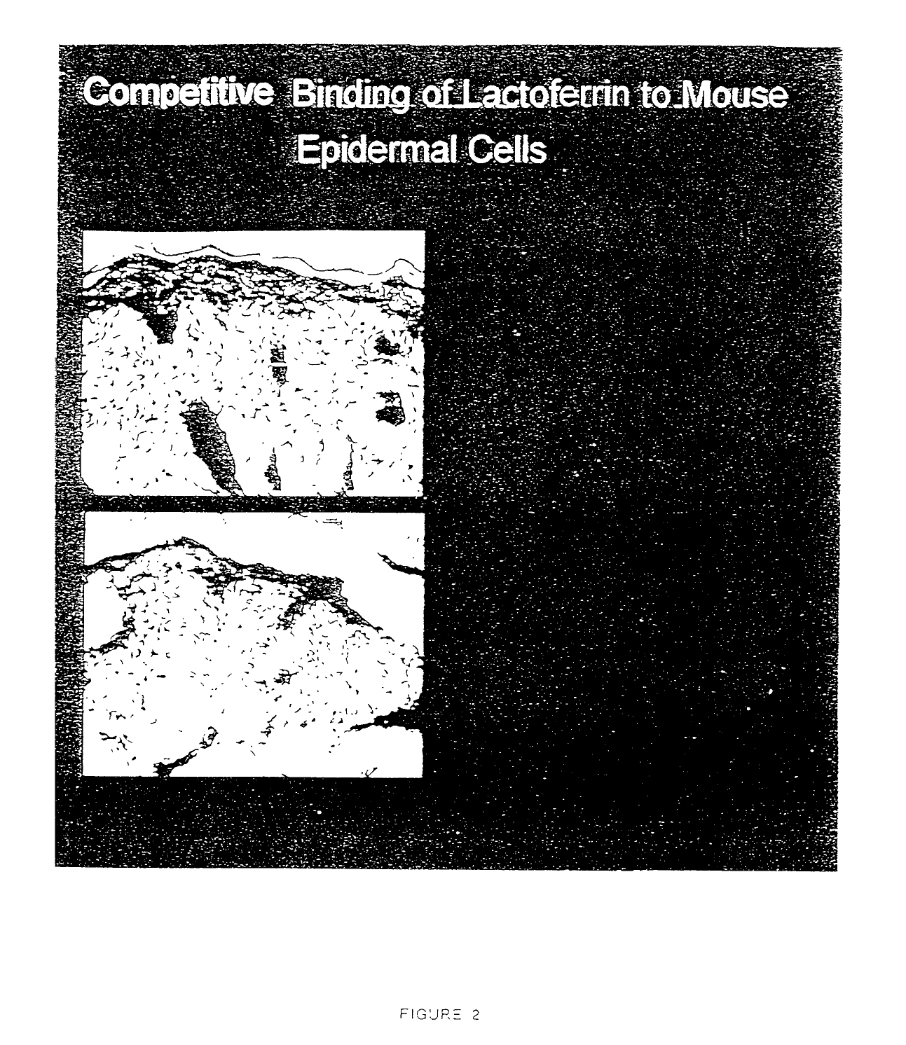

Further, it is demonstrated herein that lactoferrin binds to receptors on keratinocytes, indicating that binding of lactoferrin to keratinocytes prevents the production of TNF-α. Specifically, FIG. 2 demonstrates the binding of lactoferrin to its receptors on TNF-α producing keratinocytes in situ on neonatal mouse skin. The binding of lactoferrin is concentrated in the epidermal layer and in the sweat glands, but not in the normal epidermal cells.

The experimental results disclosed herein demonstrate that the inhibitory effect of lactoferrin on Langerhans cell migration and dendritic cell accumulation is due to the negative regulation of TNF-α production, rather than via an influence on another aspect of Langerhans cell function. Thus, lactoferrin is able to inhibit the de novo synthesis of TNF-α by binding on receptors on keratinocytes.

The evolution of a two lobe structure has endowed lactoferrin with unique iron binding properties and contributed to other biological functions of the molecule. Based on identification of those domains, sequences and structures in the lactoferrin polypeptide contributing to the iron binding properties of the protein are described in U.S. Ser. No. 08/866,544 filed May 30, 1997, the disclosure of which is herein incorporated by reference. This application provides guidance for the design and generation of novel lactoferrin variants or portions thereof having a modified iron binding capacity. Typically, lactoferrin variants have improved properties, including, but not limited to, lactoferrin variants with higher affinity for iron for improved antimicrobial activities, lactoferrin variants with lower affinity for iron having improved iron-releasing properties, or lactoferrin variants having modified pH or temperature requirements or ranges for the binding and/or release of iron. Likewise, lactoferrin variants useful in the methods of the present invention typically provide enhanced inhibition of interleukin activity such as that demonstrated by IL-1β. In addition, the invention allows for the design of lactoferrin variants having otherwise improved characteristics, e.g., therapeutic tolerance, immunoreactivity, or biological half life, while retaining their biological activity.

The lactoferrin variants of the invention may be derived from wild-type lactoferrin of a variety of mammalian species, including, but not limited to, human, murine, rate, bovine, and porcine lactoferrin. The wild-type lactoferrin may be mutated by a variety of methods generally known in the art. See, among other places, Sambrook et al., 1990 Molecular Cloning; A Laboratory Manual. Cold Spring Harbour Laboratory Press, New York; Kunkel et al., 1987, Meth. Enzymol. 154:367-382; Kunkel, 1985, Proc. Natl. Acad. Sci. USA 82:488-42.

In a preferred embodiment, the lactoferrin variants of the present invention comprise at lease one mutation in the amino acid sequence. In another preferred embodiment, the lactoferrin variants of the present invention comprise a truncated amino acid sequence.

The nucleic acid sequences encoding lactoferrin and variants thereof according to the present invention may be inserted in a vector suitable for expression in a eukaryotic cell in such way that allows expression of the lactoferrin variant. Alternatively, nucleic acid sequences encoding portions of the lactoferrin variants of the invention may be inserted in vectors allowing their expression in eukaryotic cells.

In another preferred embodiment, lactoferrin is produced in a recombinant expression system. See, e.g., Ward et al., 1992, Biotechnology 10:784-789; Ward et al., 1995, Biotechnology 13:498-503. For this purpose, nucleic acids coding for the desired form of lactoferrin (see, e.g., U.S. Pat. No. 5,571,691, incorporated by reference in its entity) is incorporated expressibly in a cellular host, which is then cultured under conditions appropriate for expression of that particular peptide or protein. A variety of gene expression systems have been adapted for this purpose, and typically drive expression of the desired gene from expression controls used naturally by the chosen host.

Because the lactoferrin variants of the invention typically, as natural occurring lactoferrin, requires post-transnational modifications, such as glycosylation at several amino acid residues, many of the lactoferrin variants or portions thereof need to be produced in an eukaryotic host. In preferred embodiments, the lactoferrin product is produced by an Aspergillus expression system, as described in Ward et al., 1992, Gene 122:219-223; and U.S. Pat. Nos. 5,571,896 and 5,571,697, the disclosures of which are herein incorporated by reference in their entirety.

If unglycosylated forms of lactoferrin variants or portions thereof are produced, however, their production may conveniently be achieved in bacterial hosts such as E. Coli. For such production, a nucleic acid coding for the selected lactoferrin variant or portion thereof, may usefully be placed under an expression control, e.g., of the lac, trp or PL genes of E. coli.

As an alternative to expression of nucleic acid coding for the lactoferrin variant or portion thereof per se, the host can be adapted to express the lactoferrin product as a fusion protein in which the lactoferrin product is linked releasably to a carrier protein that facilitates isolation and stability of the expression product.

In a further alternative, the lactoferrin variant or portion thereof may be generated by organic synthesis. In particular where production of a portion of alactoferrin variant, e.g., a peptide of about twenty (20) through about fifty (50) amino acids in length, is the objective preferably the well established techniques of automated peptide synthesis are employed, generally descriptions of which appear, for example, in J. M. Stewart and J. D. Young, Solid Phase Peptide Synthesis, 2nd Edition, 1984, Pierce Chemical Company, Rockford, Ill.; and in M. Bodanszky and A. Bodanszky, The Practice of Peptide Synthesis, 1984, Springer-Verlag, New York; Applied Biosystems 430A Users Manual, 1987, ABI Inc., Foster City, Calif.; and Solid Phase Peptide Synthesis. A practical Approach, by: E. Atherton & R. C. Sheppard, IRL Press, Oxford (1989). In these techniques, the lactoferrin variant portion is grown from its C-terminal, resin-conjugated residue by the sequential addition of appropriately protected amino-acids, using either the Fmox or tBox protocols.

Pharmaceutical Compositions Containing Lactoferrin

Lactoferrin And Lactoferrin Products. The term “lactoferrin” refers collectively herein to naturally or recombinantly produced forms of lactoferrin, particularly mammalian forms unless otherwise specified. The term “lactoferrin products” as used herein refers collectively to lactoferrin and functional analogs and functional fragments thereof which may be useful to practice the invention. Specifically, the term encompasses, for example, truncated lactoferrin and lactoferrin having one or more amino acids substituted or deleted. “Functional analogs and functional fragments” of lactoferrin refer to lactoferrin mutants and derivatives, and lactoferrin derived peptides, respectively, which have the capacity to inhibit production of TNF-α in a cell. Generally, the lactoferrin, functional lactoferrin analogs and functional fragments thereof encompassed by the invention are capable of inhibiting an inappropriate or exaggerated local immune response that involves the production of TNF-α.

Lactoferrin analogs or lactoferrin derived peptides can be tested for identification of functional analogs or functional fragments by, for example, the mouse model described herein in Example 2. See also, Cumberbatch and Kimber, 1992, Immunology 75:257-263. Briefly, this testing involves induction of a local immune response by administering an allergen, e.g., oxazolone on the shaved skin of mice. One hour later, mice are injected intradermally at the oxazolone treated site with about 0.002% to about 0.5% lactoferrin analog or fragment to be tested in saline. A second and a third group of animals are injected with lactoferrin and BSA, respectively, at the same range of concentrations and in the same buffer. After about 12 hours, the animals may be killed, and the draining lymph nodes recovered. Comparison of the numbers of accumulated dendritic cells, which may be determined as described in Cumberbatch and Kimber, supra, will reveal the efficacy of a lactoferrin analog or fragment and its usefulness for the compositions and methods of the present invention.

Generally, any substitution, addition or deletion of lactoferrin that does not destroy its receptor mediated inhibitory effects on the production of TNF-α as part of an inappropriate or exaggerated immune response may be usefully employed in this invention. Those of ordinary skill in the art can make such substitutions, addditions and deletions without undue experimentation. In preferred embodiments, the functional lactoferrin analogs or functional fragments are substantially as effective as native human lactoferrin. In the most preferred embodiments, the functional lactoferrin analogs or functional fragments have enhanced TNF-α downregulating activity compared with native human lactoferrin. For example, such functional analogs or functional fragments may exhibit enhanced serum stability, enhanced receptor binding and enhanced signal transducing activity. Other modifications to lactoferrin and functional lactoferrin analogs and functional fragments that may usefully be employed in this invention are those which render the molecule more readily bioavailable to sites of inflammation. For example, in cases where the lactoferrin product is administered topically, the functional lactoferrin analog or functional fragment may exhibit enhanced percutaneous absorption.

The particular lactoferrin products according to the present invention may be prepared by a variety of techniques well known for generating protein products. Those forms of lactoferrin that occur naturally can of course be obtained by extraction from the natural source, e.g., mammalian, most preferably human, milk, using an appropriate combination of protein isolation techniques. For example, as described in Cheron et al., 1977, C.R. Acd. Sci. Paris 284:585-588, lactoferrin isolation is achieved by sequential extraction and purification with Sephadex G-25, G-50, G-75, and G-100.

As an alternative to extraction, those forms of lactoferrin that incorporate only L-amino acids may be produced reproducibly and in commercial quantities by application of recombinant DNA technology. See, e.g., Ward et al., 1992, Biotechnology 10:784-789; Ward et al., 1995, Biotechnology 13:498-503. For this purpose, nucleic acids coding for the desired form of lactoferrin (see, e.g., U.S. Pat. No. 5,571,691, incorporated by reference in its entity) may be incorporated expressibly in a cellular host that is then cultured under conditions appropriate for expression of that particular peptide or protein. A variety of gene expression systems have been adapted for this purpose and typically drive expression of the desired gene from expression controls used naturally by the chosen host.

Because lactoferrin in its natural form requires post-translational modifications, such as glycosylation at several amino acid residues, many of the lactoferrins, functional analogs, or functional fragments thereof according to the present invention need to be produced in a eukaryotic host. In preferred embodiments, the lactoferrin product may be produced by an Aspergillus expression system, as described by Ward et al., 1992, Gene 122:219-223; and U.S. Pat. Nos. 5,571,896 and 5,571,697, herein incorporated by reference in their entirety.

If unglycosylated forms, functional analogs, or functional fragments of lactoferrin are used, however, their production may conveniently be achieved in bacterial hosts such as E. coli. For such production, a nucleic acid encoding the selected lactoferrin, functional analog, or functional fragment thereof may be placed under an expression control of, e.g., of the lac, trp or PL genes of E. coli.

As an alternative to expressing a nucleic acid encoding the lactoferrin, functional analog, or functional fragment thereof per se, a host may be adapted to express the lactoferrin product as a fusion protein in which the lactoferrin product is linked releasably to a carrier protein that facilitates isolation and stability of the expression product.

In a further alternative, the lactoferrin product may be generated by organic synthesis. In particular where the lactoferrin product is a functional fragment, e.g., a peptide of about twenty (20) through about fifty (50) amino acids in length, the well established techniques of automated peptide synthesis are employed. General descriptions of exemplary peptide syntheses appear for example, in J. M. Stewart and J. D. Young, Solid Phase Peptide Synthesis, 2nd Edition, 1984, Pierce Chemical Company, Rockford, Ill.; and in M. Bodanszky and A. Bodanszky, The Practice of Peptide Synthesis, 1984, Springer-Verlag, New York; Applied Biosystems 430A Users Manual, 1987, ABI Inc., Foster City, Calif.; and Solid Phase Peptide Synthesis—A Practical Approach, by: E. Atherton & R. C. Sheppard, IRL Press, Oxford (1989). In these techniques, the lactoferrin fragment may be grown from its C-terminal, resin-conjugated residue by the sequential addition of appropriately protected amino acids, using either the Fmoc or tBoc protocols.

In an alternative approach, functional lactoferrin fragments useful for the methods and compositions of the present invention may be obtained by peptidase digest or hydrolysis of a purified lactoferrin or lactoferrin products. Of course, lactoferrin fragments may also be produced by recombinant expression in a suitable host as set forth, supra. The invention also encompasses the use of chemically modified lactoferrin products, including methylations, carboxylation, etc.

Pharmaceutical Formulation And Routes Of Administration. For therapeutic use, the lactoferrin product(s) according to the present invention may be administered by any conventional means available for use in conjunction with pharmaceuticals, either as individual therapeutic agents or in a combination of therapeutic agents. Each can be administered alone but is generally administered with a pharmaceutical carrier selected on the basis of the chosen route of administration and standard pharmaceutical practice. The pharmaceutical compositions of the invention may be adapted for oral, parenteral, topical or rectal administration, or as an inhalant, and may be in unit dosage form, in a manner well known to those skilled in the pharmaceutical art. Parenteral administration includes but is not limited to, injection subcutaneously, intravenously, intraperitoneally or intramuscularly.

Treatment with the active ingredient may begin at any time after the indication to be treated, e.g., psoriasis, contact dermatitis, UV-induced inflammation, infant diaper rash, asthma, arthritis, and the like, is diagnosed. Preferably, treatment is commenced as a prophylactic or at early stages of the disease, in order to prevent massive inflammation in the first place. Typically, treatment will continue until the inflammation is cured. In cases of chronic diseases, such as psoriasis, asthma or arthritis, or in cases of continued exposure to an allergen, the treatment may have to be extended beyond the cure of the symptoms. Because lactoferrin is a naturally occurring non-toxic protein, side effects are not expected even in cases of a long-term treatment.

The dose administered will, of course, vary depending upon known factors, such as (1) the pharmacodynamic characteristics of the particular lactoferrin product and its mode and route of administration, (2) the age, health, height and weight of the recipient, (3) the nature and extent of the symptoms, (4) the kind of concurrent treatment(s), (5) the frequency of treatment(s), and (6) the effect desired. A daily dose of active ingredient can be expected to be about 1 milligram to about 1.2 grams per kilogram of body weight, with the preferred dose being 50 milligrams to about 500 milligrams per kilogram of body weight. When the active ingredient is administered topically to the skin or in an inhalant, the active ingredient is typically applied in 0.005% to about 5% admixed with the carrier, preferably being about 0.05% to about 0.5% admixed with the carrier.

Dosage forms (compositions suitable for administration) contain from about 0.005% to about 0.5% of active ingredient per unit. In these pharmaceutical compositions, the active ingredient is ordinarily present in an amount of about 0.5-95% by weight based on the total weight of the composition.

Typically, the active ingredient will be administered topically, as inhalant, or as injection in inflamed joints or cartilage. However, alternatively the lactoferrin products may be administered orally in solid or semi-solid dosage forms, such as hard or soft gelatin capsules, tablets, or powders, or in liquid dosage forms, such as elixirs, syrups, or suspensions. It can also be administered parenterally, in sterile liquid dosage forms. Other dosage forms are potentially possible such as patches or ointment or transdermal administration.

The lactoferrin products of the invention may also be formulated as a slow release implantation device for extended and sustained administration of the lactoferrin product. Examples of such sustained release formulations include composites of biocompatible polymers, such as poly(lactic acid), poly(lactic-co-glycolic acid), methylcellulose, hyaluronic acid, collagen, and the like. The structure, selection and use of degradable polymers in drug delivery vehicles have been reviewed in several publications, including, A. Domb et al., Polymers for Advanced Technologies 3:279-292 (1992). Additional guidance in selecting and using polymers in pharmaceutical formulations can be found in the text by M. Chasin and R. Langer (eds.), “Biodegradable Polymers as Drug Delivery Systems,” Vol. 45 of “Drugs and the Pharmaceutical Sciences,” M. Dekker, New York, 1990. Liposomes may also be used to provide for the sustained release of a lactoferrin product. Details concerning how to use and make liposomal formulations of drugs of interest can be found in, among other places, U.S. Pat. No. 4,944,948; U.S. Pat. No. 5,008,050; U.S. Pat. No. 4,921,706; U.S. Pat. No. 4,927,637; U.S. Pat. No. 4,452,747; U.S. Pat. No. 4,016,100; U.S. Pat. No. 4,311,712; U.S. Pat. No. 4,370,349; U.S. Pat. No. 4,372,949; U.S. Pat. No. 4,529,561; U.S. Pat. No. 5,009,956; U.S. Pat. No. 4,725,442; U.S. Pat. No. 4,737,323; U.S. Pat. No. 4,920,016. Sustained release formulations are of particular interest when it is desirable to provide a high local concentration of a lactoferrin product.

Gelatin capsules or liquid-filled soft gelatin capsules may contain the active ingredient and powdered or liquid carriers, such as lactose, lecithin starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar-coated or film-coated to mask any unpleasant taste and to protect the tablet from the atmosphere, or enteric-coated for selective disintegration in the gastrointestinal tract. Liquid dosage forms for oral administration can contain coloring and/or flavoring to increase patient acceptance.

Suitable pharmaceutical carriers are further described in Remington's Pharmaceutical Sciences 17th ed., Mack Publishing Company, Easton, Pa. (1990) a standard reference text in this field, which is incorporated herein by reference in its entirety.

In preferred embodiments, pharmaceutical compositions comprising lactoferrin products may be administered topically for the treatment of allergic skin disorders, as an inhalant for the treatment of pulmonary inflammatory diseases such as asthma, in the form of a nasal spray for rhinits and sinusitis, or in the form of injections for the treatment of arthritis. Besides the lactoferrin product, these compositions may comprise additional active components, including, but not limited to, hydrocortisone, retinoic acid, or conventional adjuncts used for current topical therapies. The preferred pharmaceutical dosage forms for the administration of the lactoferrin products of this invention can be illustrated as follows:

Topical Formulation

In cases where treatment of inflammatory skin disorders is desired, the preferred route of administration will typically be topical. As lactoferrin is secreted, e.g., by sweat glands (Masson et al., 1966, Clin. Chem. Axta 14:735-739), lactoferrin products may generally be expected to enter through pores of the skin when administered topically. The availability of the active substance to its site of action will be further alleviated by the fact that inflammation is typically accompanied by disruption of the surface of the skin. Alternatively, substances which enhance the penetration through the skin may be added to the formulation.

Generally, for topical administration, the lactoferrin products may be formulated as a solution, gel, lotion, ointment, cream, suspension, paste, liniment, powder, tincture, aerosol, transdermal drug delivery system, and the like in a pharmaceutically acceptable form by methods well known in the art. Actual methods for preparing topical formulations are known or apparent to those skilled in the art, and are described in detail in Remington's Pharmaceutical Sciences 17th ed., Mack Publishing Company, Easton, Pa. (1990); and Pharmaceutical Dosage Forms and Drug Delivery Systems, 6th ed., Williams & Wilkins (1995).

In order to enhance the percutaneous absorption of the active ingredients, a number of agents may be added in topical formulations, including, but not limited to, dimethylsulfoxide, dimethylacetamide, dimethylformamide, surfactants, azone, alcohol, acetone, propylene glycol and polyethylene glycol. In addition, physical methods may also be used to enhance transdermal penetration such as iontophoresis or sonophoresis.

The pharmaceutical compositions may be applied directly to the skin. Alternatively, they may be delivered by various transdermal drug delivery systems, such as patches.

Formulations for Inhalants

In particular for the treatment pulmonary inflammatory diseases such as asthma and bronchitis, compositions comprising the lactoferrin product will typically be administered as an inhalant.

Generally, the lactoferrin product may be administered using a conventional inhaler. The lactoferrin product may be administered alone, or it may be co-administered with other inhalation aerosols, including, but not limited to, beta-adrenergic agonists, e.g., albuterol inhalation aerosols, salmeterol xinafoate inhalation aerosols, terbutaline sulfate inhalation aerosols; adrenocortical steroids, e.g., beclomethasone diproprionate inhalation aerosols; antiasthmatic, antiallergic, or mast cell stabilizers, e.g., cromolyn sodium inhalation aerosols; antiinflammatory aerosols, e.g., triamcinolone acetonide topical aerosol; anticholinergic agents, e.g., ipratropium bromide inhalation aerosols; or sympathomimetic substances, e.g., isoetharine mesylate inhalation aerosols, and metaproterenol sulfate inhalation aerosol. For further reference, additional teachings in these regards are provided in Pharmaceutical Dosage Forms and Drug Delivery Systems, by: Ansel et al., Williams & Wilkins, PA (1995).

Formulations for Sprays

In particular for the treatment of rhinitis, sinusitis and sunburn, compositions comprising the lactoferrin product will typically be administered as a spray.

Generally, the lactoferrin product may be administered using a conventional spray device such as those commercially available. The lactoferrin product may be administered alone, or it may be co-administered with other substances, including, but not limited to antihistamines, sympathomimetic agents and antibiotics. Moreover, the lactoferrin product may be administered in medicated sprays such as sodium chloride nasal solution (Salinex solution (Muro)) or Xylometazoline hydrochloride nasal solution (Sine-Off (SmithKline Beecham)) or functionally analogous sprays. For further reference, additional teachings in these regards are provided in Pharmaceutical Dosage Forms and Drug Delivery Systems, supra.

Formulations for Injections

In particular for the treatment of arthritis and cellulitis, the compositions comprising a lactoferrin product may be administered by injection, e.g., locally into the diseased joint or cartilage, or systemically.

In general, water, oil, saline, aqueous dextrose (glucose), polysorbate and related sugar solutions and glycols such as propylene glycol or polyethylene glycols are suitable carriers for parenteral solutions. Solutions or emulsions for parenteral administration preferably contain about 5-15% polysorbate 80 or lecithin, suitable stabilizing agents, and if necessary, buffer substances. Antioxidizing agents, such as but not limited to, sodium bisulfite, sodium sulfite, or ascorbic acid, either alone or combined, are suitable stabilizing agents. Also used are citric acid and its salts and sodium EDTA. In addition, parenteral solutions can contain preservatives, including but not limited to, benzalkonium chloride, methyl- or propyl-paraben, and chlorobutanol.

Further suitable pharmaceutical carriers for parenteral administration are described in Remington's Pharmaceutical Sciences, supra.

Indications

As discussed above, TNF-a plays important roles in the pathogenesis and/or progression of a variety of acute and chronic inflammatory disease processes. As a consequence, there has been considerable interest in designing synthetic inhibitors of TNF-α production that may be used therapeutically. The evidence cited herein indicates that lactoferrin products provide a means for regulating TNF-α production. The potential therapeutic applications of lactoferrin that derive from these data are many and include, but are not limited to, the treatment of the following discussed inflammatory disorders in a mammal, in particular a human in need.

Contact Dermatitis

Contact dermatitis is an eczematous dermatitis caused by exposure to substances in the environment. Those substances act as irritants or allergens and may cause acute, subacute, or chronic eczematous inflammation. Irritant contact dermatitis, i.e., irritation of the skin, is the most common form of contact dermatitis. Mild irritants may cause dryness, fissuring, and erythema, when exposure is continuous. For example, continuous exposure to moisture in areas such as the hand, the diaper area, e.g., infant diaper rash, or the skin around a colostomy may eventually cause eczematous inflammation. Strong chemicals may produce an immediate reaction. See, T. P. Habif, in: Clinical Dermatology, Mosby, Mo. (1996) and The Textbook of Medicine, edts: J. B. Wyngaarden and L. H. Smith, W., W. B. Saundres Company, Philadelphia (1985).

Allergic contact dermatitis, in contrast to irritant contact dermatitis, is a delayed hypersensitivity reaction that affects a limited number of individuals after one or a few exposures to a substance exhibiting antigenic activity on that particular individual.

Both irritant and allergic contact dermatitis are believed to be triggered by a local immune cascade involving de novo expression of TNF-α in keratinocytes as explained, supra.

Current Treatment Methods And Their Drawbacks. Acute contact dermatitis is currently treated with cold wet dressings, accompanied by the administration of steroids, e.g., hydrocortisone, prednisolone, methylprednisone, and, in severe cases, fluorinated corticosteriods, which are powerful anti-inflammatory agents at very low concentrations. Less potent than the fluorinated steroids, hydrocortisone, prednisolone and methylprednisone are used for less severe cases. Depending on the severity of the inflammation, the steroids are applied topically or systemically.

However, many adverse effects have been reported for steroids, especially in the case of the powerful fluorinated steroids. For example, epidermal and dermal atrophy can be a pronounced adverse effect and decreased collagen synthesis and reduced stromal support for blood vessels may lead to telangiectasia, purpura, and striae. A perioral dermatitis has also been reported with the fluorinated steroids, as well as aggravation of facial erythema, restricting absolutely the extended use of these compounds for the face. Further, possible elevated intraocular pressure warrants a strong proscription against the prolonged application of any topical steroids near the eyes.

The reality of systemic absorption of topical steroids presents an additional hazard. Lowering of plasma cortisol level is seen with as little as 20 per cent of the body under occlusion. The risk of rebound after discontinuing steroids in those skin diseases characterized by the phenomenon cannot be overlooked.

Overall, a need for low-toxicity treatment methods for contact dermatitis is apparent.

Improved Treatment Methods Provided By The Present Invention. The present invention provides novel pharmaceutical compositions comprising a lactoferrin product for the treatment of contact dermatitis. In a preferred embodiment, the compositions are formulated for topical application. Specifically, the lactoferrin product may be applied in the form of lotions, creams, ointments and the like, in concentrations of about 0.005% to about 5%, in preferred embodiments in concentrations of about 0.01% to about 0.5% active substance. Typically, the composition will comprise adjuncts conventionally used for topical administration. See, supra. In alternative embodiments, the compositions are formulated for intradermal injections. The compositions of the present invention directly interfere with the production of TNF-α by keratinocytes, thus preventing the exaggerated local immune response which is the underlying cause of irritant as well as allergic contact dermatitis. Since lactoferrin is known to be a non-toxic protein, the compositions provided will have non-detectable side effects.

Psoriasis

Psoriasis is a chronic inflammatory skin disorder that undergoes repeated relapse and remission and affects one (1) to three (3) percent of the world's population. In the United States more than one million people have psoriasis, and of these more than eleven (11) percent have experienced disability of such severity as to compromise employment and effectiveness. The disease can begin at any age and has its peak appearance in the third decade. Its severity, course, and remissions are unpredictable. See, T. P. Habif, in: Clinical Dermatology, Mosby, Mo. (1996) and The Textbook of Medicine edts: J. B. Wyngaarden and L. H. Smith, W., W. B. Saundres Company, Philadelphia (1985).

Patients with psoriasis can be divided into two groups, those with a genetic predisposition and those that respond to epigenetic, i.e. environmental factors. The most common form is chronic plaque psoriasis and is characterized by hyperplasia of the epidermis and inflammation of the dermis and epidermis. These changes arise due to activation of a T-lymphocyte cell mediated dermal immune reaction in regional lymph nodes in response to unidentified antigenic stimuli. The activated T-cells cause keratinocyte cells to proliferate and assume a psoriasis phenotype. The inflammatory reaction is caused by proinflammatory cytokine proteins that are induced in response to the environmental stimulus and results in infiltration of the dermis and epidermis by inflammatory white blood cells.

Current Therapeutic Strategies And Their Drawbacks. There is no cure for psoriasis and therapeutic strategies currently in use seek to suppress the severity and extent of the disease. The first line of therapy for patients with <10% of body affected is a topically administered antiinflammatory agent. The most widely used agents in the United States are corticosteriods (hydrocortisone and analogs) because of their high degree of short-term efficacy and relatively low cost. Systemic corticosteriods, for example, can induce prompt resolution of psoriatic lesions, but suppression requires ever-increasing doses. When therapy is tapered, there is a rebound phenomenon, with extension of lesions possibly to exfoliation. If systemic steroids are used for other valid reasons in patients with psoriasis, the risk of aggravated psoriasis should be recognized and weighed in the decision for initiating therapy.

While psoriasis is responsive to treatments which can induce remission for a time period, with repeated administration tolerance develops to the antiinflammatory action of the treatment and benefit does not persist more than a few months. Further, serious side effects that involve pituitary-adrenal suppression have been observed in infants when standard over-the-counter dosages of hydrocortisone (1%) are used. James et al., 1967, Lancet 2:1059-1061; Turpeinen, 1991, Br. J. Dermatol. 124:358-360. Further side effects of steroids have been addressed, supra.

In sum, there is a clear need for treatment methods of psoriasis, which are effective while having less severe side effects.

Improved Treatment Methods Provided By The Present Invention. Recent advances in our understanding of the central role of immune reactions in the pathogenesis of psoriasis has led to an intense investigation of the application of immunotherapy to alleviate the disease. Specifically, psoriasis has been shown to be caused by inappropriate local immune responses which involve the de novo production of TNF-α by keratinocytes. Gilhar et al., 1996, Clin. Exp. Immunol. 106: 134-142. Accordingly, in one embodiment, the present invention provides compositions that directly interfere with the production of TNF-α by keratinocytes, thus preventing the inappropriate local immune response causing psoriasis. More specifically, the present invention provides novel pharmaceutical compositions comprising a lactoferrin product for the treatment of psoriasis. In a preferred embodiment, the compositions are formulated for topical application in about 0.5% to about 5% carrier. In alternative embodiments, the compositions are formulated for intradermal injections.

As addressed above, psoriasis is believed to be an incurable disease, thus may require long-term therapy. One important advantage of the compositions of the present invention over current treatment methods is thus the low-toxicity (or non-toxicity) of the lactoferrin-products.

UV-Induced Inflammation

Exposure to UV light is another predominant source of local skin reactions and diseases, including sunburn photoaging of the skin, and cancer.

Current Preventive Treatment Methods And Their Drawbacks. Currently, sunscreens with photo-protectives, e.g., p-aminobenzoic acid, as active ingredients are applied to reduce penetration of photoactive nonionizing radiation to the viable epidermal cells beneath the keratin, i.e., to prevent the adverse reactions to UV radiation. These photoprotectives are typically designed to protect against the shorter burning rays of ultraviolet light in the wavelength range of 290 to 320 nm, UVB. Sun screens thus provide an incomplete block to permit melanin production relative to the radiation transmitted and the inherent capacity of the partially protected skin to respond with tanning.

A number of inflammatory skin reactions, as well as skin aging, however, are evoked by longer burning rays in the range of 320 to 400 nm, UVA, against which the typical photoprotective does not provide protection. If protection is desired against UVA, then physical sunscreens are needed such as titanium dioxide, or zinc oxide, which are available as heavy creams or pastes. However, these opaque protectors are not nearly as acceptable as the clear and milky lotions that protect against UVA, and furthermore they do not allow for a self-protective tanning reaction of the skin. See, T. P. Habif, in: Clinical Dermatology, Mosby, Mo. (1996) and The Textbook of Medicine, edts: J. B. Wyngaarden and L. H. Smith, W., W. B. Saundres Company, Philadelphia (1985).

Current Curative Treatment Methods And Their Drawbacks. Once UV-induced skin inflammation, i.e., sunburn, has occurred, it is usually treated with cold compresses and time. More severe cases are treated with prednisone, which may cause severe side effects, such as epidermal and dermal atrophy, further telangiectasia, purpura, and striae as explained, supra.

In short, there is a need for substances which both prevent and/or cure UV5 induced inflammation of the skin.

Improved Preventive And Curative Treatment Methods Provided By The Present Invention. The present invention provides improved compositions and methods for the treatment and the prevention of UV-induced skin irritation. More specifically, in one embodiment, the invention provides novel UV protective compositions comprising a conventional sunscreen basis and an inflammation-preventive lactoferrin product as an additive. These novel pharmaceutical compositions are prone to prevent UV-induced skin inflammation, including UVA induced-inflammation, while allowing tanning of the skin.

In an alternative embodiment, the invention provides pharmaceutical compositions comprising a lactoferrin-product for the treatment of UV-induced skin inflammation.

Photoaged Skin

As discussed above, one of the adverse effects of exposure to UV light is aging of the skin, also referred to as photoaging.

Current Treatment Methods Of Photoaged Skin With Tretinoin And Its Drawbacks. Currently, topical application of tretinoin provides some reversal of photodamaged skin. Weiss et al, 1988, J. AMA 259:527-532. However, most patients experience tretinoin-induced dermatitis as a side-effect, which may endure for weeks to months. See, T. P. Habif, in: Clinical Dermatology, Mosby, Mitsouri (1996) and The Textbook of Medicine, edts: J. B. Wyngaarden and L. H. Smith, W., W. B. Saundres Company, Philadelphia (1985). Thus, there is a need for improved products for the treatment of photoaged skin.

Improved Compositions Provided By The Present Invention. The present invention provides improved compositions and methods for the treatment of photoaged skin. More specifically, in one embodiment, the invention provides conventional tretinoin compositions comprising a lactoferrin product as protective against the a tretinoin-induced inflammatory side effects.

Antiwrinkle Products

In recent years a number of “antiwrinkle” cosmetic products for topical application have entered the market. These products, frequently used by individuals who want to prevent facial skin aging, or even reverse the aging process which has already occurred, contain hydroxyacids as active ingredients. While these products indeed seem to have the desired “de-wrinkling” activity, they frequently cause inflammation of the skin as a side effect. Accordingly, there is a need for modifications to the currently used antiwrinkle products that abate their inflammatory side effects.

Improved Compositions Provided By The Present Invention. According to the present invention, lactoferrin products may be added to conventional antiwrinkle creams, lotions, and other products, as an additive to protect against hydroxyacid-induced inflammatory side effects.

Acne

Another disorder caused or associated with inflammation of the skin is acne, a skin disorder which interferes with the quality of life of many individuals. See, T. P. Habif, in: Clinical Dermatology, Mosby, Mo. (1996) and The Textbook of Medicine, edts: J. B. Wyngaarden and L. H. Smith, W., W. B. Saundres Company, Philadelphia (1985).

Current Treatment Methods Of Photoaged Skin With Tretinoin And Its Drawbacks. For lighter forms of the disease, antimicrobial preparations, e.g., benzoyl peroxide, or clindamycin, tetracycline, or erythromycin are frequently prescribed for topical application.

Alternatively, the disease may be treated with systemic broad-spectrum antimicrobials. However, though more effective topical therapy, such treatment methods frequently do not result in the desired success, and furthermore, they are associated with serious side effects. For example, tetracyclines, although commonly used, carry a risk of dental discoloration, photosensitivity, and even unexpected pregnancy through drug interaction with oral contraceptives. Because they are incorporated into growing bones and teeth, they should not be prescribed from the fourth fetal month through age 12. There is also the possibility of subsequent mobilization from bone under stress, as in pregnancy, with consequent risk of maternal hepatitis or fetal absorption. Erythromycin is a useful alternative, however, it frequently results in gastrointestinal distress.

More severe acne may require more extensive surgical procedures, higher doses of antimicrobials, intralesional steroids, or even systemic steroids. Estrogens have been used in selected women with stubborn severe acne with good results, but used as cyclic estrogen progestin therapy they all have the attendant risk of anovulatory preparations given for contraception.

In sum, current treatment methods for acne usually leave the patient without a satisfactory cure, while having rather severe side effects. Thus, there is a need for effective low toxicity therapeutics.

Improved Compositions Provided By The Present Invention. The present invention provides effective compositions comprising a lactoferrin product for the therapy of inflammation associated with acne. Generally, the compositions are formulated similarly as those used for the treatment of contact dermatitis.

Arthritis

Arthritis, i.e., the inflammation of cartilage of joints, is associated with a number of causes and is known to have a variety of manifestations. For example, arthritis may be an autoimmune disorder, or, alternatively, it may be induced by an allergen. See, The Textbook of Medicine, edts: J. B. Wyngaarden and L. H. Smith, W., W. B. Saundres Company, Philadelphia (1985).

Current Treatment Methods And Their Drawbacks. Currently, arthritis is typically treated systemically with corticosteroids or other steroids. The problems related with steroid treatment are discussed, supra. Furthermore, none of the current treatment methods for arthritis may stop the progression of the disease, which may result in deformation of the skeleton. Thus, there is a demanding need for improved compositions and methods for the treatment of arthritis.

Improved Compositions Provided By The Present Invention. The present invention provides novel compositions comprising a lactoferrin product which inhibit allergen-mediated inflammation useful for the treatment of many forms of arthritis. Furthermore, as the mediation of an autoimmune response appears to rely on similar mechanisms as allergen related immune responses, i.e., both are TNF-α mediated, it can be assumed that the compositions provided also will be of value for the treatment of forms of arthritis related to autoimmune disorders. In one embodiment of the invention, compositions comprising a lactoferrin product as an active ingredient are directly injected into diseased joints. Other anti-inflammatory agents, such as salicylates, may be used as adjuncts. In other embodiments of the invention, lactoferrin products may be administered systemically by injection, or in the form of sustained release products. An advantage of the compositions of the present invention over any previously known products for arthritis therapy are (1) effectiveness, directly interfering with the driving force of the disease, i.e., inflammation of the joints, thereby preventing progression of the disease, and (2) non-toxicity, i.e, as pointed out, supra, lactoferrin products have been shown to exhibit no toxic effects on humans and other mammals.

Pulmonary Inflammatory Diseases

Pulmonary Inflammatory Diseases such as allergic asthma and bronchitis are disorders characterized by increased responsiveness of the trachea and bronchi to various stimuli, i.e. allergens, resulting in widespread narrowing of the airways. For instance, asthma is an inflammatory reaction of the respiratory system in response to external stimuli. In severe cases, asthma may cause death through lack of oxygen supply and may afflict as many as five per cent of the population in the United States. In over half the cases, asthma is diagnosed between ages of two (2) and seventeen (17) years, and in this group it is the leading cause of disease and disability. See, The Textbook of Medicine, edts: J. B. Wyngaarden and L. H. Smith, W., W. B. Saundres Company, Philadelphia (1985).

Current Treatment Methods And Their Drawbacks. Current treatment methods include the administration of sympathomimetic drugs, methylxanthines, and corticosteroids. Sympathomimetic drugs, including epinephrine and isoproterenol, have a beta-adrenergic effect. Their usefulness is limited by their actions on the heart, furthermore, tolerance develops after repeated use. Methylxanthines, which are believed to cause smooth muscle relaxation by their action on the cytoplasmic enzyme phosphodiesterase, in many cases cause anorexia, nausea, gastrointestinal upset, and central nervous system irritability. Corticosteroids are very effective in the treatment of asthma, however, they have severe side effects as discussed, supra. Thus, there is a need for new compositions and methods for the treatment of asthma which are effective while having low toxicity.

Improved Compositions Provided By The Present Invention. The present invention provides new compositions comprising a lactoferrin product and methods for the treatment of asthma. Typically, the compositions will be administered as an inhalant. Alternatively, compositions comprising the lactoferrin product may be administered systemically by injection or in the form of tablets, capsules, or in sustained release forms. Even at high dosage, the compositions of the invention will not have significant side effects, because lactoferrin is a natural, non-toxic protein.

The below examples explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawing. Such modifications are intended to fall within the scope of the appended claims.

### EXAMPLES OF THE PREFERRED EMBODIMENTS

### Example 1

Binding of Lactoferrin to Receptors Located on TNF-α Producing Keratinocytes

The following example is an in situ ligand-binding assay showing the binding of ¹²⁵J-lactoferrin to keratinocytes in the epidermal layer of neonatal mouse skin.

Experimental Procedure. Approximately 2 mm² back-skin samples from newborn wild type mouse were obtained and rinsed briefly twice with PBS at 37° C. and incubated by duplicate at 37° C. in 0.5 ml of PBS/0.1% bovine serum albumin contained:

(A) 1.0×10^{8 125}I-lactoferrin; and

(B) 1.0×10^{8 125}I-lactoferrin plus 50 folds excess unlabeled iron saturated recombinant lactoferrin.

Samples were washed six (6) times for ten (10) minutes each in cold PBS/0.1% bovine serum albumin and fixed in 4% paraformaldehyde for three (3) hours without shaking and then rinsed twice for ten (10) minutes in cold PBS. Immediately the samples were frozen. The samples were used to make 10 μm sections, and then the sections were dehydrated using ascending concentrations of ethanol. Sections were placed in NTB-2 emulsion and exposed for seven (7) and fourteen (14) days at 4° C. After development the sections were stained with hematoxylin and mounted with Permount.

Binding Of Lactoferrin To Keratinocytes. As the in situ hybridization experiment reveals, lactoferrin accumulates at the membrane of keratinocytes. See e.g., FIG. 2. Specifically, FIG. 2 shows the binding of lactoferrin to its receptors on TNF-α producing keratinocytes in situ on neonatal mouse skin. The top two panels show the bright and the dark field images obtained using only labelled lactoferrin (1.0×10^{8 125}I-lactoferrin). The bottom two pabels show the bright and the dark field images when the binding assay was done in the presence of excess (50-fold excess) unlabelled lactoferrin. The binding of lactoferrin is concentrated in the epidermal layer and in the hair follicles, but is not observed in the normal dermal layer. These results indicate the binding of lactoferrin to its receptors on the surface of keratinocytes in the epidermis. Keratinocytes are known to produce TNF-α.

### Example 2

Influence of Lactoferrin on DC Accumulation in Draining Lymph Nodes

The following example shows that lactoferrin inhibits the accumulation of dendritic cells in draining lymph nodes induced by oxazolone and IL-1β, while lactoferrin does not affect the accumulation of dendritic cells induced by administration of TNF-α.

Experimental Procedure. Groups of mice (n=10) received 30 μl intradermal injections into both ear pinnae of either 0.02% murine lactoferrin (LF) or 0.02% bovine serum albumin (BSA) each suspended in phosphate buffered saline (PBS; pH 7.2). Two (2) hours later, the mice received either a second intradermal injection at the same site of 50 ng (30 μl) of cytokine, i.e., TNF-α or IL-1β, suspended in 0.1% BSA/PBS, or were exposed topically on the dorsum of both ears to 25 μof 0.5% oxazolone (Ox) dissolved in acetone:olive oil (4:1). Control mice were untreated. Draining (auricular) lymph nodes were removed at various times following exposure dependent upon kinetics of induced DC accumulation for each treatment, i.e., four (4) hours for TNF-α, seventeen (17) hours for IL-1β, and eighteen (18) hours for oxazolone, respectively. Subsequently, the number of dendritic cells per node was assessed as described previously. Cumberbatch and Kimber, 1992, Immunology 75 :257-263.

Results. As depicted in TABLE I and FIG. 3A, treatment with oxazolone induced the accumulation of dendritic cells in draining lymph nodes (bar 4, in comparison to the control group depicted as bar 1). Intradermal administration of lactoferrin to the oxazolone-induced animals, however, resulted in a inhibition of accumulation of dendritic cells in the draining lymph nodes by 75% (Oxazolone/lactoferrin: 13,774 dendritic cells per node (above baselevel), oxazolone/+lactoferrin: 3,394 dendritic cells per node (above baselevel), see, TABLE I), indicating suppression of a local immune response in these animals by lactoferrin.

As further illustrated in TABLE I and FIG. 3A, administration of TNF-α induced accumulation of dendritic cells in draining lymph nodes (see, FIG. 3A, bar 2). As indicated by the results reflected by bar 3 of FIG. 3A, lactoferrin does not have any effect on the TNF-α induced local immune response, i.e., administration of TNFα/−lactoferrin resulted in 4,753 dendritic cells per node (above baselevel), while administration of TNF-α/+lactoferrin resulted in 4,802 dendritic cells per node (above baselevel). These results indicate that lactoferrin acts upstream of TNF-α in the immune response cascade.

As depicted in TABLE I (B) and as further illustrated in FIG. 3B, intradermal administration of IL-1β resulted in stimulation of Langerhans cell migration and dendritic cell accumulation in draining lymph nodes (FIG. 3B, bar 1). The administration of lactoferrin resulted in an inhibition of the immune response. As indicated in FIG. 3B, bar 2, intradermal injection of lactoferrin resulted in inhibition of dendritic cell accumulation by 56% (IL-1β/−lactoferrin: 7,569 dendritic cells per node (above baselevel), oxazolone/+lactoferrin: 3,342 dendritic cells per node (above base level), see, TABLE I).

These results indicate that:

(1) dendritic cell accumulation is dependent on increases in IL-1β levels, and

(2) IL-1β acts upstream of TNF-α, since a TNF-α induced immune response is not affected by lactoferrin.

Thus, IL-1β stimulates the de novo synthesis of TNF-α by keratinocytes. Furthermore, these data demonstrate that the inhibitory effect of lactoferrin on Langerhans cell migration and dendritic cell accumulation is due to the negative regulation of TNF-α production rather than via an influence on another aspect of Langerhans cell function. Thus, lactoferrin is able to inhibit the de novo synthesis of TNF-α.

TABLE I
INFLUENCE OF LACTOFERRIN ON DC ACCUMULATION
IN DRAINING LYMPH NODES

| | |
|---|---|
| | DC/Node |
| Treatment | Above Base |

| | −2 hours | 0 hours | DC/Node | Level |
|---|---|---|---|---|
| A | — | — | 3313 | |
| | BSA | TNF-α | 8066 | 4753 |
| | LF | TNF-α | 8115 | 4802 |
| | BSA | Ox | 17087 | 13774 |
| | LF | Ox | 6707 | 3394 |
| B | — | — | | |
| | BSA | IL-1β | 10882 | 7569 |
| | LF | IL-1β | 6655 | 3342 |
| | BSA | Ox | 17213 | 13900 |
| | LF | Ox | 8911 | 5598 |

### Example 3

Influence of Lactoferrin on TNF-α- and IL-1β-Induced Langerhans Cell Migration

The following example demonstrates that lactoferrin inhibits IL-1β-induced Langerhans cell migration, while it does not affect migration of Langerhans cells which is induced by TNF-α.

Experimental Procedure. Groups of mice (n=3) received 30 μl intradermal injections in both ear pinnae of either 0.005% murine lactoferrin (LF) or 0.005% bovine serum albumin (BSA) each suspended in phosphate buffered saline (PBS; pH 7.2). Two hours later mice received a second intradermal injection at the same site of 50 ng (30 μl) of cytokine, i.e., TNF-α or IL-1β, respectively, suspended in 0.1% BSA/PBS. Control mice were untreated. Ears were removed 30 mins following TNF-α treatment or 17 hours following IL-1β treatment and epidermal sheets prepared as described previously. Cumberbatch et al., 1994, Immunology 81:395-401. The frequency of Langerhans cells (LC) was measured by indirect immunofluorescence. Results are expressed as the mean number of cells/mm³ (±SE) derived from examination of 10 fields/sample for each of 4 samples. The statistical significance of differences between experimental groups was calculated using the Student's t-test. p.<0.005.

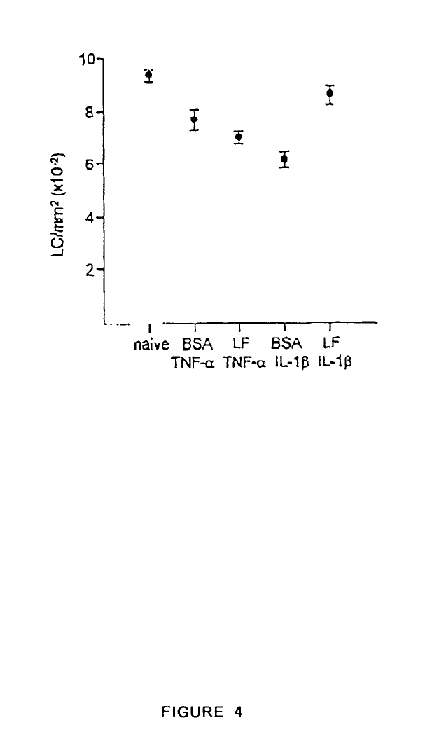

Results. As depicted in TABLE II and FIG. 4, intradermal administration of IL-1β resulted in stimulation of Langerhans cell migration, which was inhibited by lactoferrin. Also administration of TNF-α resulted in migration of Langerhans cells, however, TNF-α induced migration was not inhibited by lactoferrin.

These results confirm that IL-1β acts upstream of TNF-β in the local immune response cascade, and that lactoferrin acts downstream of IL-1β, but upstream of TNF-α.

TABLE II
INFLUENCE OF LACTOFERRIN ON TNF-A AND IL-β—INDUCED
LANGERHANS CELL MIGRATION.

| | |
|---|---|
| Treatment | Langerhans |

| −2 hours | 0 hours | Cells per mm² |
|---|---|---|
| — | — | 938.9 ± 22.2 |
| BSA | TNF-α | 766.5 ± 36.9 |
| LF | TNF-α | 701.2 ± 21.2 |
| BSA | IL-1β | 615.6 ± 16.1 |
| LF | IL-1β | 858.9 ± 28.9 |

### Example 4

Influence of Lactoferrin on the Accumulation of Dendritic Cells in Draining Lymph Nodes

The following example shows that lactoferrin, when administered topically, inhibits the accumulation of dendritic cells in draining lymph nodes induced by oxazolone.

Experimental Procedure. Groups of mice (n=6) received topically a 0.02% solution of lactoferrin dissolved in lubriderm lotion or 0.02% bovine serum albumin (BSA) dissolved similarity on the dorsum of both ears. Later, the mice were exposed topically at the same sites to 25 ul of 0.5% oxazolone (Ox) dissolved in acetone:olive oil (4:1). Control mice were untreated. Draining lymph nodes were removed 18 hours following exposure and the number of dendritic cells per node was assessed as described previously. Cumberbatch et al., 1992, Immunology 75:257-263.

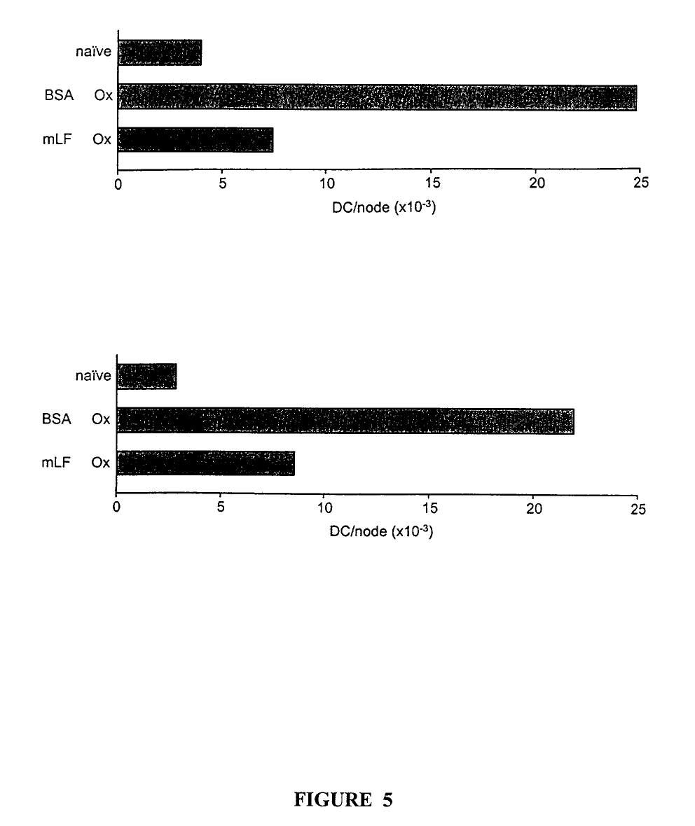

Results. As depicted in FIG. 5, results from two independent experiments (panels A and B) indicate that treatment with oxazolone induced accumulation of dendritic cells in draining lymph nodes of mice pretreated with 0.02% BSA (see bar 2 panels A and B versus bar 1 control). Topical administration of mouse lactoferrin resulted in a strong inhibition of accumulation of dendritic cells in the lymph nodes in response to oxazolone in both experiments (see bar 3, panels A and B).

The results indicate that topically administered lactoferrin is highly effective at inhibiting dendritic cell accumulation in lymph nodes in response to an allergen and hence allergen-induced cutaneous inflammation.

FIG. 5 shows the inhibitory effect of lactoferrin on oxazolone induced dendritic cell accumulation in draining lymph nodes when lactoferrin is applied topically to the skin surface.

All references cited within the body of the instant specification are hereby incorporated by reference in their entirety.

The examples provided herein are purely exemplary. The invention is intended to be limited only by the scope of the appended claims.

What is claimed:

1. A method of inhibiting the inflammatory activity of IL-1β comprising the step of administering a pharmaceutically effective amount of a lactoferrin product.

2. A composition comprising (a) a therapeutic or cosmetic compound that produces a local inflammatory reaction, and (b) a lactoferrin product.

3. A composition according to claim 2, wherein the compound of (a) is selected from the group consisting of tretinoin, a photoprotective, and a hydroxyacid.

4. A composition according to claim 2 or claim 3, further comprising a pharmaceutically acceptable carrier or a cosmetic carrier.

5. A method of treating an allergen-induced inflammatory disorder in a mammal, comprising the step of administering to a mammal a therapeutically effective amount of a lactoferrin product.

6. The method of claim 5, wherein the mammal is a human.

7. The method according to any one of claims 1, 5 and 6 wherein the allergen-induced inflammatory disorder features a local immune response characterized by increased production of TNF-α.

8. The method according to any one of claims 1 and 5-7 wherein the lactoferrin product is a naturally occurring lactoferrin.

9. The method according to any one of claims 1 and 5-7 wherein the lactoferrin product is a recombinantly produced lactoferrin or a biologically active analog thereof.

10. The method according to any one of claims 1 and 5-7 wherein the lactoferrin product is a biologically active fragment of lactoferrin.

11. The method according to any one of claims 1, 5, 6, 7, 8, 9 and 10 wherein the inflammatory disorder is selected from the group consisting of arthritis and a pulmonary inflammatory disease.

12. The method according to any one of claims 1, 5, 6, 7, 8, 9 and 10 wherein the inflammatory disorder is a dermal inflammatory disorder.

13. The method according to any one of claims 1, 5, 6, 7, 8, 9 and 10 wherein the inflammatory disorder is selected from the group consisting of contact dermatitis, psoriasis, UV-induced inflammation, infant diaper rash, and acne.

14. The method according to any one of claims 1, 5, 6, 7, 8, 9 and 10 wherein the lactoferrin product is administered in a composition further comprising an anti-wrinkle agent and the inflammatory disorder is facial skin aging.

15. The method according to any one of claims 1, 5, 6, 7, 8, 9 and 10 wherein the inflammatory disease is asthma.

16. The method according to any one of claims 1, 5, 6, 7, 8, 9 and 10 wherein the inflammatory disease is sinusitis.

17. The method according to any one of claims 1, 5, 6, 7, 8, 9 and 10 wherein the inflammatory disease is rhinitis.

18. The method according to any one of claims 5, 6, 7, 8, 9 and 10 wherein the inflammatory disease is bronchitis

19. The method according to any one of claims 5, 6, 7, 8, 9 and 10 wherein the inflammatory disease is bronchitis.
